(19) United States
(12) Patent Application Publication
(10) Pub. No.: US 2011/0152829 A1
(43) Pub. Date: Jun. 23, 2011

(54) Patient Fluid Management System

(75) Inventor: Yasuhiro Kawamura, Tokyo (JP)

(73) Assignee: K&Y Corporation

(21) Appl. No.: 12/972,374

(22) Filed: Dec. 17, 2010

Related U.S. Application Data
(60) Provisional application No. 61/287,881, filed on Dec. 18, 2009.
(60) Provisional application No. 61/287,903, filed on Dec. 18, 2009.
(60) Provisional application No. 61/287,912, filed on Dec. 18, 2009.
(60) Provisional application No. 61/287,991, filed on Dec. 18, 2009.

(51) Int. Cl. A61M 5/172

(52) U.S. Cl. 604/503; 604/67; 604/66; 604/500

(57) ABSTRACT

A patient fluid care system that provides an integrated solution for managing patient fluids by precisely controlling one or more infusion pumps; monitoring real-time patient sensors, and pharmaceutical information; and deriving patient status and the status of pharmaceuticals administer to the patient based upon such monitoring and controlling; and automatically adjusting infusing parameters based upon the real-time patient, infusion, and pharmaceuticals information and derived patient status and the pharmaceuticals levels.

### RELATED APPLICATIONS

This application claims priority to U.S. Provisional Application Ser. No. 61/287,881 filed Dec. 18, 2009, entitled MEMS Pump for Medical Infusion Pump; U.S. Provisional Application Ser. No. 61/287,903 filed Dec. 18, 2009, entitled Pump Stay; U.S. Provisional Application Ser. No. 61/287,912 filed Dec. 18, 2009, entitled Micro Infusion Pump System Software; and U.S. Provisional Application Ser. No. 61/287,991 filed Dec. 18, 2009, entitled Central Venous Pressure Monitoring Using Micro Infusion Pump, the contents of which are each incorporated in their entirety herein.

### FIELD OF THE INVENTION

The present invention relates to patient fluid management systems and related methods and, more particularly, to patient fluid management systems employing disposable piezoelectric driven infusion pumps.

### BACKGROUND OF THE INVENTION

Medical treatments commonly involve providing pharmaceuticals, vitamins, nutrients, metabolism products and the like to patients through what is referred to as infusion pumps. Infusion pumps administer fluids directly into the blood, into the body tissues, the digestive tract, the respiratory system, the mucous membranes, or the skin of a patient. Furthermore, patient treatment often includes simultaneously administering multiple fluids to a single patient. In turn, the types of fluids and the flow rates and total volumes of the fluids to be administered are determined by the patient's particular health issue, the patient's vital signs, and other real-time patient biological data obtained from various patient monitors.

Accordingly, physicians and other medical staff are often presented with the challenge of gathering needed pharmaceuticals, medical equipment, and patient biological data from various, independently managed, and often remotely located data sources. Various forms of multi-component infusion systems have been proposed, such as those described in U.S. Pat. Nos. 4,756,706 to Kerns et al; 4,898,578 to Rublacaba; 5,256,157 to Samiotes et al.; and 5,713,856 to Eggers et al. However, these systems fail to provide fully integrated solutions to the above described challenges faced by medical staff. For example, the Egger patent describes an overly complicated system that relies upon two-way communication between the infusion pump and an interface unit; i.e. the infusion pumps of Egger communicate data back to the interface unit.

Accordingly, in order to improve the safety and accuracy of infusion treatments, an integrated system is needed for precisely driving delivery of infusion fluids while simultaneously obtaining patient biological information and providing medical staff with real-time infusion status and related projections regarding a patient's fluid and pharmaceuticals status. Furthermore, in order to more efficiently utilize medical staff and to provide enhanced patient safety, a system is needed that can react or adjust operating specifications within ranges established by the medical staff, such as fluid flow rates and total infusion volumes, based on real-time patient sensor inputs.

### OBJECTS AND SUMMARY OF THE INVENTION

A patient fluid care system according to the present invention provides a precise delivery of infusion fluids while simultaneously obtaining patient biological information and providing medical staff with real-time infusion status and related projections regarding a patient's fluid and pharmaceuticals status. Furthermore the patient fluid care system according to the present invention can react to or adjust a predetermined infusion protocol or plan within ranges established by the medical staff according to real-time patient information.

One aspect of the present invention provides real-time displays of various patient and infusion parameters. Another aspect of the present invention provides real-time patient fluid balance and pharmaceutical level information to the medical staff. In yet another aspect of the present invention, the present invention automatically adjusts a patient's predetermined infusion protocol or plan based upon real-time patient biological data received during infusion.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings.

FIG. 1 is a perspective view of a patient fluid care system according to one embodiment of the present invention.

FIG. 2 is a perspective view of an infusion pump of a patient fluid care system according to one embodiment of the present invention.

FIG. 3 is a schematic of the data flow and processing of a patient fluid care system according to one embodiment of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

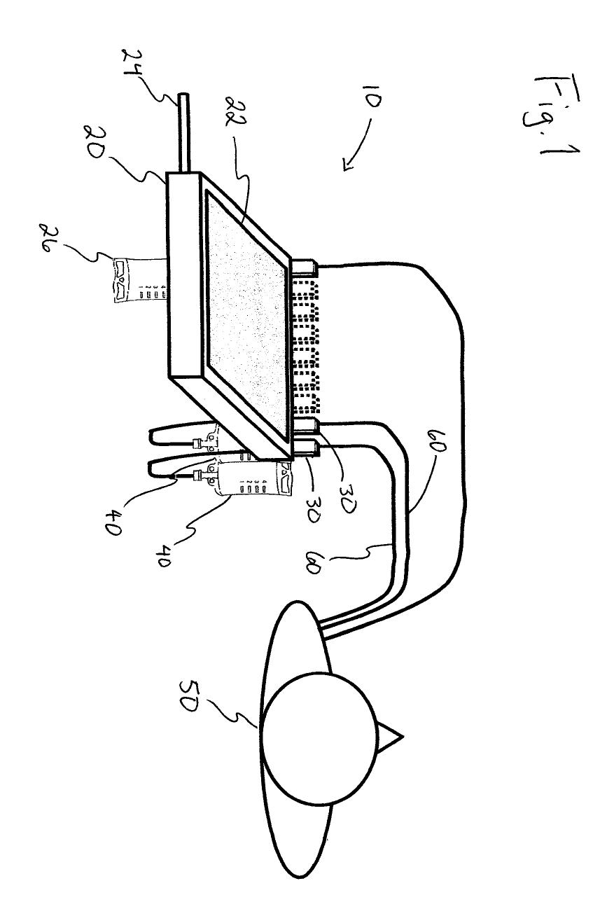

Patient fluid care systems according to the present invention provide a single interface from which a patient's nutritional or pharmacological fluids are managed and monitored. As shown in FIG. 1, a patient fluid care system 10 according to the present invention comprises a terminal 20 having a user interface 22. The user interface 22 may, for example, comprise a touch screen display. The terminal 20 serves to control one or more infusion pumps 30 which function to provide an infusion fluid from a fluid reservoir 40 to the patient 50 through patient line 60. The patient line 60 may comprise a variety of different tubing and catheter systems known in the field. The terminal 20 may further comprise an intravenous fluid post or stand for supporting infusion fluid reservoirs or bags 40. Preferably, the intravenous fluid post or stand is height adjustable.

With respect to the infusion pump 30, it is contemplated that a variety of types of infusion pumps, including peristaltic pumps, syringe pumps, and elastomeric pumps, can be employed as the infusion pump 30. In order to achieve the greatest accuracy and convenience, it is preferred that the infusion pump 30 be a microelectromechanical, or MEMS, micropump driven by a piezoelectric effect. In brief, such micropumps can be fabricated using known integrated circuit fabrication methods and technologies. For example, using integrated circuit manufacturing fabrication techniques, small channels can be formed on the surface of silicon wafers. By attaching a thin piece of material, such as glass, on the surface of the processed silicon wafer, flow paths and fluid chambers can be formed from the channels and chambers. A layer of piezoelectric material, or a piezoelectric body such as quartz, is then attached to the glass on the side opposite the silicon wafer. When a voltage is applied to the piezoelectric body, a reverse piezoelectric effect, or vibration, is generated by the piezoelectric body and transmitted through the glass to the fluid in the chamber. In turn, a resonance is produced in the fluid in the chamber of the silicon wafer. Through the inclusions of valves and other design features in the fluid flow paths, a net directional flow of fluid through the chamber formed by the silicon wafer and the glass covering can be achieved. Examples of such pumps and related control systems are described in greater detail in the Assignee's copending U.S. Patent Application No. (TBD) entitled Infusion Pump, the contents of which are herein incorporated in their entirety.

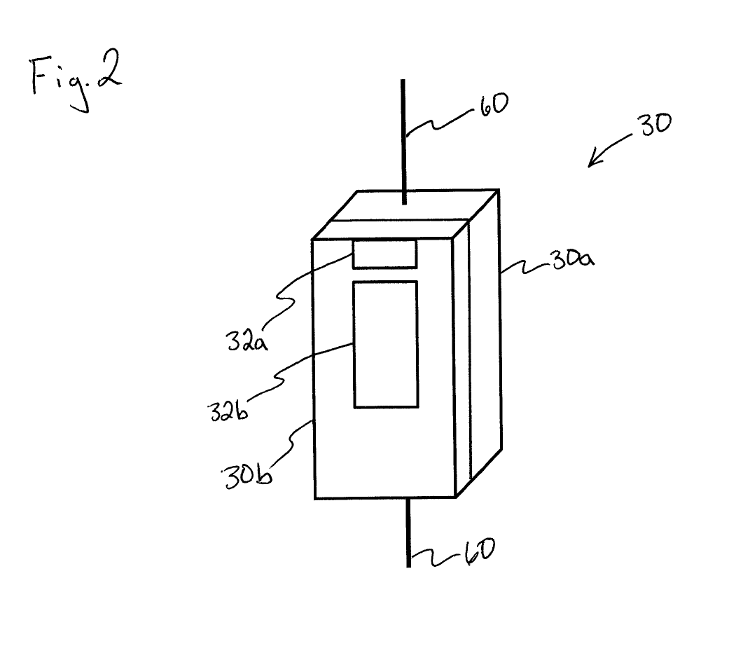

As shown in FIG. 2, according to one embodiment of the present invention, the infusion pump 30 comprises a disposable body 30a and a non-disposable or reusable base 30b. The disposable body 30a houses components of the infusion pump 30 that contact the infusion fluid, e.g. the piezoelectric micropump, a flow meter, not shown, and fluid inlet and outlet ports, also not shown. The base 30b of the infusion pump 30 houses the control circuitry for providing power to the pump 30 and the sensing circuitry for providing flow meter data back to the terminal 20. The disposable body 30a and the reusable base 30b of the infusion pump 30 are configured to mate with one another thereby creating a single pump unit 30 and establishing electrical communication between the body 30a and the base 30b.

The base 30b may be separate from or incorporated into the terminal 20. In applications in which the base 30b is not integrated into the terminal 20, the base 30b comprises one or more electrical connectors, not shown, for establishing electrical communication with the terminal 20. Furthermore, applications in which the base 30b is not integrated into the terminal 20, the base 30b comprises a physical mount, not shown, that is complementary to a second mount that is either integrated into the terminal 20 or operable to be attached to a support structure, such as an intravenous fluid post or stand.

In certain embodiments, as shown in FIG. 2, the base 30b of the infusion pump 30 further comprises one or more user interfaces 32a and 32b. The interface 32a may comprise a visual indicator, such as a light, positioned on the base 30b so as to face the user and thereby provide warnings or alerts to the user. The interface 32b may comprise a display that, for example, displays the name, abbreviation, or other indicator of the fluid being pumped through the same pump 30.

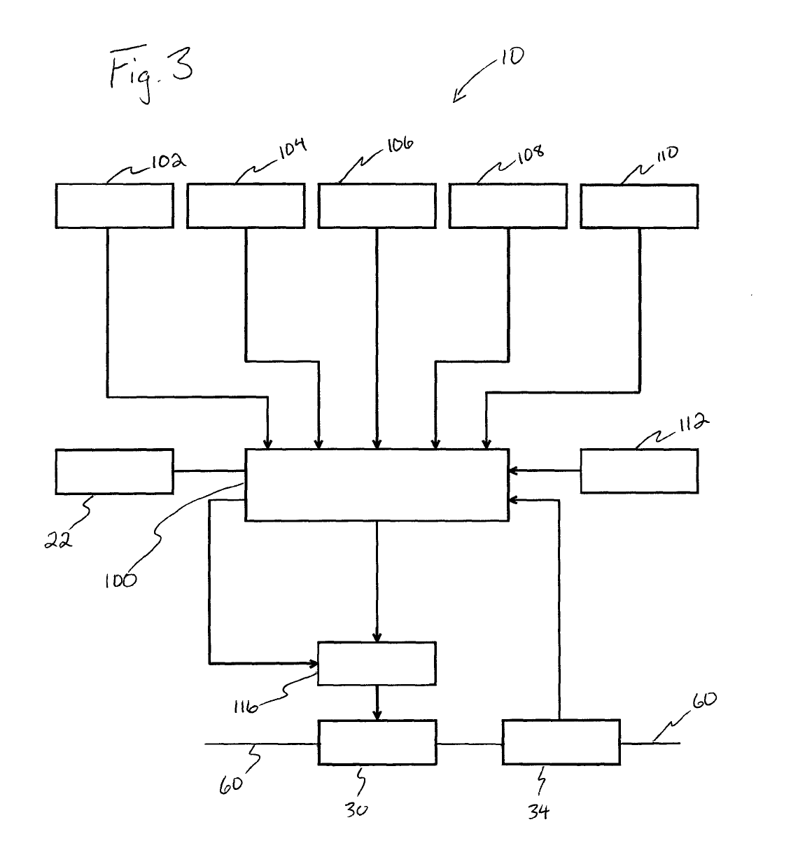

The patient fluid care system 10 further comprises a flow meter 34, shown in FIG. 3, that functions to measure actual infusion fluid flow throw the patient line 60 downstream of the infusion pump 30. The flow meter 34 may comprise a variety of known flow meters. For example, the flow meter 34 may be configured to determine fluid flow rates by employing a heater that heats the fluid being monitored and senses the flow of the heated fluid downstream of the heater. Such flow meters are available from Sensirion AG of Switzerland and Siargo Incorporated of the United States of America and are described in greater detail in at least U.S. Pat. No. 6,813,944 to Mayer et al. and U.S. Publication No. 2009/0164163, which are herein incorporated by reference. Alternatively, the flow meter 34 may be configured to employ two pressure sensors positioned on each side of a constriction within the fluid flow path. Fluid flow rates are determined by the relative difference between the pressure sensors and changes thereof. The flow meter 34 is preferably integrated into the infusion pump 30, for example within the base 30b.

Broadly speaking, in operation and with reference to FIG. 1, the terminal 20 determines and provides control factors for operation of the infusion pumps 30 based upon inputs entered into the terminal 20 via the user interface 22 or via a data network connector 24. The data network connector 24 may, for example comprise a LAN, local area network, connector in data communication with a user interface at a remote location. Such remote locations may, for example, be a nursing station and/or dispensary, or doctors' room. Data network capability also facilitates use of the patient fluid care system 10 according to the present invention for homecare market.

In a preferred embodiment, the control factors provided to the infusion pump 30 by the terminal 20 are determined by a combination of user inputs; sensor inputs received by the terminal 20 from one or more patient sensor; and data obtained from one or more pharmaceutical product databases or libraries. Each of these inputs will be described in greater detail below. It will be noted that in certain medical situations, it may be desirable to administer multiple infusion fluids, e.g. nutritional and pharmaceutical fluids, simultaneously to a single patient. It is contemplated that the patient terminal 20 according to the present invention, can monitor the infusion conditions for a plurality of patient lines 60, for example up to thirty patient lines 60, and can physically support and control the operation of a plurality of infusion pumps 20, for example up to thirty infusion pumps 20.

FIG. 3 is a schematic diagram showing the functionality and workflow of the patient fluid care system 10 according to one embodiment of the present invention. The arrows indicate the direction or flow of data. Housed preferably within the patient terminal 20 is a central processing unit or CPU 26 that is associated with one or more data memory units; a power supply circuitry for providing power to the infusion pump and various patient sensors; and a sensor data circuitry to receive patient sensor data from various patient sensors. Note that for the sake of clarity, the patient terminal 20 is not specifically shown in FIG. 3.

As shown in FIG. 3, patient data 102 is provided to the CPU 100, for example, by manually entering the data through the user interface 22 and/or by a bar code reader associated with the patient terminal 20. Patient information 102 may comprise a patient's name, a patient's identification number, a patient's contact information, a patient's emergency contact, a patient's caregiver's name; a patient's caregiver's contact; and insurance provider information.

Patient biological data is provided to the CPU 100, for example, by manually entering the data through the user interface 22 and/or by patient sensor inputs. Patient biological data comprises, for example, a patient's weight, blood pressure, insensible perspiration, urinary output, as well as a patient's other vital signs. In this regard, the CPU 100 is configured to be in real-time data communication with various patient sensors and monitors such as, a circulatory pressure meter 104 and a scale 106 for determining the weight or mass of a patient's urine collected in a urine bag 26, shown in FIG. 1. It will be understood that a wide range of other patient sensors may be associated with the patient fluid care system 10 of the present invention. For example the patient care system 10 may be associated with patient temperature sensors, heart rate sensors, and various sensors for measuring the oxygenation of a patient's hemoglobin, such as a pulse oximeter. Furthermore, a patient's physician may input or delineate patient biological data pertaining to a patient's fluid or water balance, as well as define patient fluid balance thresholds.

Patient pharmacological data 108 is provided to the CPU 100, for example, by manually entering the data through the user interface 22 and/or by a bar code reader associated with the patient terminal 20. The patient pharmacological data 108 may comprise data regarding the common, brand, or scientific name of a pharmaceutical to be administered to a patient, and the desired dose of the pharmaceutical.

In certain embodiments of the present invention data from a pharmacological database 112 is also provided or accessed by the CPU 100. The pharmacological database 112 may be permanently or temporarily stored in a memory component associated with the patient fluid care systems 10 or may be accessed at a remote location through the network connector 24 of the patient terminal 20. The pharmacological database 112, for example, may provide pre-determined standard dosages; maximum toxic dose, or MTD, which is the dosage above which toxicity may arise; minimum effective dose, or MED, which is the dose below which there is no effect; metabolic rates, e.g. the half-life of the pharmaceutical within the body, and other pharmacokinetic information.

Infusion data 110 is provided to the CPU 100, for example, by manually entering the data through the user interface 22. Infusion data 110 may comprise data regarding the specific infusion plan or protocol in which the pharmaceutical or pharmaceuticals provided as patient pharmacological data 108 is administered to the patient through the infusion pump 20 of the patient fluid care systems 10. Specific infusion data 110, for example, may comprise specifying a specific infusion pump 20, a flow rate of the infusion, a total volume of infusion fluid to be infused. Related to the Infusion data 110 are the particular control factors 116 that direct operation of the infusion pump 30 including, for example, the voltage provided to the pump 30, the frequency at which the voltage is applied to the pump 30, and the rates at which the voltage and frequency are increased or decreased. It will be understood that the control factors 116 may not be directly provided by a user through the user interface 22 as the infusion data 110 but rather determined by the CPU 100, or rather a software module of the CPU 100, based upon the infusion data 110.

In one embodiment of the patient fluid care systems 10 according to the present invention, patient medical history data is provided to the CPU 100 and thereby accessible from the user interface 22 of the patient terminal 20. Such patient medical history may, for example, be accessed from an electric medical record, or EMR, system through the network connector 24 of the patient terminal 20.

It is noted that that the CPU 100 and associated memory components of the patient fluid care systems 10 of the present invention includes data input logs that provide for the retrieval and review of current and previously obtained patient and pharmaceutical data. The CPU 100 may further comprise software modules operable to conduct certain comparisons and calculations as will be described in greater detail below.

Based upon the inputs described above and the continual monitoring of such inputs, the patient fluid care system 10 according to the present invention provides the user with real-time information relating to the state of a patient. Such real-time information would typically only be available to a caregiver, such as a physician, through independent and often remote medical equipment and information resources. One advantage of the present invention is the consolidation and presentation of such medical information in single patient terminal or, by means of the network data compatibility, at location remote from the patient.

An additional advantage of the patient fluid care systems 10 according to the present invention is the operability of the system 10 to analyze the above-described data inputs in order to derive additional real-time patient information. For example, the patient fluid care system 10 calculates and provides real-time patient drug levels to the patient's caregiver based upon certain of the inputs relating to the patient biological data, the patient pharmacological data 108; the pharmacological database 112; and the infusion data 110 described above. More particularly, the CPU 100 may comprise a pharmaceutical level software module that by monitoring patient vital signs, real-time infusion volumes, and taking into consideration the pharmacokinetics of the specific drug or drugs being administered, the patient fluid care system 10 derives real-time estimates regarding the pharmaceuticals administered their quantities present in a patient's body.

Central to the accuracy of the drug level calculations is the integration of patient specific data such as a patient's weight; infusion specific data, such as the actual flow rate and volume of the each of the pharmaceuticals being infused into the patient; and the pharmacokinetics relevant to each of the pharmaceuticals being infused into the patient that is accessed from the pharmacological database 112. Furthermore, based upon integration of the system 10 with the pharmacological database 112, the system 10 can compare infusion plans entered in to the system 10 by a caregiver against known pharmacological information and based upon the comparison, provide warnings or alerts where potential errors may have been made during input of the infusion plan.

Yet another example of the operability of the patient fluid care system 10 to analyze the above-described data inputs in order to derive additional real-time patient information is the system operability to calculate patient water or fluid balances. For example, the CPU 100 may comprise a fluid balance software module that provides caregivers with patient water balance information by utilizing the patient's urinary output data which is determined by data received from the scale 106 that continually weighs the patient's urine bag, the infusion data regarding the volume of water and other fluids infused into the patient up to that time; and the patient's volume of insensible perspiration.

The water management information derived by the patient fluid care system 10 can be used by physicians in determining subsequent patient treatments. For example, in medical settings such as those for premature infants in which infusion volumes must be closely monitored due to the patient's small size, the concentration of pharmaceutical products are selected on the basis of the infant's water balance data, i.e. the amount of urine and insensible perspiration lost and the total infusion quantity input. Furthermore, a patient's fluid balance is of significant importance during central venous hyperalimentation in which high volumes of infusion fluids can potentially place hazardous loads on a patient's heart and kidneys.

In one embodiment of the present invention, the patient fluid care system 10 is also operable to utilize the above described data inputs and calculated patient pharmaceutical levels and fluid balance in order to automatically adjust patient infusion protocols or plans within the parameters originally set by the physician. The CPU 100 may comprise a compensation software module to determine and implement these automatic adjustments. More particularly, in operation, the physician or caregiver will enter in to the patient fluid care system 10 the patient's infusion plan, e.g. the volume of the pharmaceutical to be administered and the flow rate at which the pharmaceutical will be administered. Additionally, the physician may set certain parameters and stipulations within which the infusion plan will be administered. If, for example, central venous hyperalimentation is being administered, the physician may enter an initial infusion flow rate and final infusion volume that are not to be exceeded. The physician may further instruct the system 10 to monitor a patient's urinary output and if during infusion, the urinary output is below a set threshold, the infusion will automatically adjust infusion flow rates or terminate infusion.

In yet another example, a patient's blood pressure may be managed by administering catecholamines and beta blockers, which are counterbalancing pharmaceutical products. In this case, blood pressure is controlled by administering multiple drugs that are often antagonistic having different mechanisms of action. The patient fluid care system 10 allows the physician to set up infusion plans that will automatically administer such antagonistic pharmaceuticals based upon inputs from a patient sensor such as blood pressure. The automatic control can be performed, including stipulating antagonistic pharmaceuticals within a range of infusion and biological sensor inputs determined by the medical staff. Furthermore, through access to the pharmacological database 112, the patient fluid care system 10 can execute MTD and MED warnings or alarms to notify medical staff.

In one embodiment of the present invention, the patient fluid care systems 10 further comprises a simulation function. The simulation function, among other tasks, estimates patient fluid balances at a projected time during infusion, for example, at the time infusion of a pharmaceutical is contemplated to start.

The patient fluid care system 10 of the present invention is particularly advantageous in that while at a patient's bedside, a physician may access and view pages displaying data compilations through the patient terminal 20 and the software functionality of the patient fluid care systems 10. Such pages may, for example, display the infusion status of each pharmaceutical being infused, including the dose remaining in the patient body; display infusion status of each pharmaceutical by grouping; and display the patient's water or fluid balance.

Furthermore, the patient fluid care system 10 of the present invention is particularly advantageous in that the micropump 30 of the infusion pump 10 does not exchange data with the patient terminal 20, i.e. the electrical communication between the micropump 30 and the patient terminal 20 is one-way, from the patient terminal 20 to the micropump 30. The micropump 30 is a slave to the patient terminal 20. This configuration is advantageous because it provides for a simplified and more economical infusion pump 10. For example, by making the micropump 30 a slave of the patient terminal 20, the circuitry within the micropump 30 is simplified and thereby more economical to manufacture. In view of the embodiments in which portions of the micropump 30 are disposable, a hospital or clinic may more economically obtain the disposable portions of the patient fluid care system 10 and only have to acquire and maintain a limited number of the more complex and more costly patient terminals 20.

The patient fluid care system 10 employing data network capabilities and a piezoelectric driven infusion pumps are especially well suited for homecare deployment because they may be configured to monitor a patient's central venous pressure without the need for conventional, costly central venous pressure monitoring equipment. This functionality is described in greater detail in the Assignee's copending U.S. Patent Application No. (TBD) entitled CIRCULATORY PRESSURE MONITORING USING INFUSION PUMP SYSTEMS, the contents of which are herein incorporated in their entirety.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A patient fluid care system comprising:
a infusion pump;
a flow meter;
a patient fluid line in fluid communication with the infusion pump and the flow meter;
a patient sensor; and
a patient terminal in data communication with the infusion pump, the flow meter and the patient sensor, the patient terminal comprising a central processing unit having an infusion compensation module that determines control factors for the infusion pump based upon data inputs from the flow meter and the patient sensor.

2. The system of claim 1 wherein the infusion pump is piezoelectric driven.

3. The system of claim 1 wherein the infusion pump comprises a disposable portion.

4. The system of claim 1 wherein the patient sensor comprises a scale.

5. The system of claim 1 wherein the patient sensor comprises the infusion pump.

6. The system of claim 1 wherein the patient terminal further comprises a patient interface.

7. The system of claim 1 wherein the patient terminal further comprises a touch screen patient interface.

8. The system of claim 1 wherein the central processing unit of the patient terminal further comprises a patient fluid balance module that determines a patient fluid balance based upon data inputs from the flow meter and the patient sensor.

9. A method for patient fluid care comprising:
defining a patient infusion plan including patient infusion thresholds;
providing an infusion fluid to a patient according to the defined patient infusion plan;
receiving patient sensor data from a patient sensor during the providing of the infusion fluid to the patient; and
adjusting automatically the patient infusion plan according to the received patient sensor data and the patient infusion thresholds.

10. The method of claim 9 wherein the step of defining a patient infusion plan comprises determining an infusion pump control factor.

11. The method of claim 9 wherein the step of providing an infusion fluid to a patient according to the defined patient infusion plane comprises pumping the infusion fluid to the patient with a piezoelectric driven infusion pump.

12. The method of claim 9 wherein the step of receiving patient sensor data from a patient sensor during the providing of the infusion fluid to the patient comprises receiving patient circulatory pressure data from an infusion pump.

13. The method of claim 9 wherein the step of receiving patient sensor data from a patient sensor during the providing of the infusion fluid to the patient comprises receiving patient urinary output data from a scale.

14. The method of claim 9 wherein the step of adjusting automatically the patient infusion plan according to the received patient sensor data and the patient infusion thresholds comprises determining a patient fluid balance.

15. The method of claim 9 wherein the step of adjusting automatically the patient infusion plan according to the received patient sensor data and the patient infusion thresholds comprises determining a pharmaceutical level in the patient.

16. The method of claim 9 wherein the step of adjusting automatically the patient infusion plan according to the received patient sensor data and the patient infusion thresholds comprises accessing a pharmaceutical database.

17. A method for patient fluid care comprising:
entering a patient infusion plan in to a patient terminal;
administering an infusion fluid to a patient according to the patient infusion plan;
receiving patient biological information during the administering of the infusion fluid;
receiving infusion flow information during the administering of the infusion fluid; and
providing a patient parameter calculated from the received patient biological information and the received infusion flow information to a user interface of the patient terminal.

18. The method of claim 17 wherein the step of administering an infusion fluid to a patient according to the patient infusion plan comprises employing a piezoelectric driven infusion pump.

19. The method of claim 17 wherein the step of providing a patient parameter calculated from the received patient biological information and the received infusion flow information to a user interface of the patient terminal comprises providing a patient fluid balance.

20. The method of claim 17 wherein the step of providing a patient parameter calculated from the received patient biological information and the received infusion flow information to a user interface of the patient terminal comprises providing a pharmaceutical level of the patient.
